SEQUENCE LISTING (6 sequences)

SEQ ID NO: 1 - DNA, 3240 bp, Synechococcus vulcanus
  CDS (169)..(3201)
gtgaattatggcgttcgcgatcgcctgcaacacctggaactgattctgagcagcaaaacagcttagaactgttaacaaatccttaatacgcctgttatcaattgttgtaccgccctggttcaggattgggtaaagtaaggagtattatctttgcaggctggggtaaatatgacatcagtcctcgatgtgaccaatcgcgatcgcttaattgaaagtgaaagtttggcagcccgtaccctacaggaacggttgcgactggtggaagaggtcttggtcgatgtcttggcggcagaatcgggtcaagaattggttgatctattgcggcgcttgggggctctctcttcgccggaaggtcatgtgctccatgccccagaaggggaattgctgaaggttattgaatccctcgaactcaatgaggccattagagcggcccgggcttttaacctctactttcaaattatcaacatcgttgagcagcattacgaacaacaatacaaccgtgaacgcgctgcccaagagggattgcgccgccgcagtgtcatgagtgaaccaatttccggtgtcagtggtgaaggctttccgctgcctcatactgctgccaacgcaacggatgtgcgcagtgggccgagtgaacgcctagagcatagtctctacgaagccattcccgctactcagcagtatggttcctttgcttggctctttcctcggctgcagatgctgaatgtgccgccgcgccatattcaaaagcttttggatcaactggacattaagttggttttcactgctcacccgacggagattgtgcggcaaacgattcgtgataagcagcggcgggttgcccgattacttgagcaactggatgtgctggagggggcttctccacacctaacggattggaacgcccaaactttacgggcacaactgatggaggaaattcgcctctggtggcgcaccgatgagttgcaccaatttaagccggaggtgctcgatgaggtggaatacaccctccactacttcaaggaggtcatttttgctgtcattcccaagctctatcgccgtctggagcagtcattacatgaaacctttcccgcgcttcagcccccccgtcaccgtttctgccgctttggctcttgggtggggggcgatcgcgatggcaatccctatgtcaaaccagaagtaacgtggcaaacggcctgctatcagcgcaacttagttcttgaggagtatattaagtccgttgagcgcttaatcaatttgctcagcctgtccctgcactggtgcgatgtgctgccagatttgctagattcccttgagcaggatcaacggcaactcccgagtatctatgagcagtatgcggtgcgctatcggcaggaaccctaccgcctgaaactggcctatgtgctcaaacggctgcaaaatacccgcgatcgcaaccgggcgctgcaaacctattgcattcgccgcaatgaggcggaagagttaaataatggacagttttaccgccacggtgaagaattcttggcagaactgctgctcattcagcgtaacctcaaggaaacgggattggcctgccgcgaattggatgatttgatttgccaggtggaggtctttggctttaatctagcagccttggatattcgccaagaaagtacctgtcacgctgaggccctcaatgaaattaccgcctatttgggtattctcccctgtccctatacagaactctcagaagccgaacgcacccgctggctcctcagtgaactctcgacccgtcgccccttgattccaggggaactcccctttagcgatcgcaccaatgaaatcattgaaacattccgcatggtgcggcaactccagcaggaatttggcaccgatttgtgcaatacctacatcatcagcatgagccatgaggtcagcgatctgttggaggtactcctctttgctaaggaggcaggcctttttgatccagccactggcgctagtaccctgcaagccattcccctgtttgaaacggtggaggatctcaagcacgccccagcggtgctgacccaactattctctctccccttttgccggagctatcttggaagcaacagtaccccctttctgcaggaggtcatgctgggctattccgacagcaataaggattcgggcttcctcagtagcaactgggaaatttataaggcacaacaacagctgcagaaaattgctgagagttttggcttccaactgcgcattttccacggtcggggtggttcagtgggtcggggtggtggacctgcctatgcggcgattttggcacagccagcacaaacgattaagggacgaatcaagattactgagcagggggaggtactggcttccaaatactcgttgccggaactcgcgctctttaacctcgaaacagtggccacagcggtcatccaagctagtttgctccgcagtagtattgatgagattgagccttggcacgagattatggaggagttggctacgcgatcgcgccagtgctatcgccatctcatctatgagcagccagaattcattgaattctttaacgaagtcaccccaatccaagagattagccaactgcaaattagctcacggccaacacggcggggggggaagaaaacccttgagagcctgcgggcaattccttgggtctttagttggacgcaaacccgtttcctgctgccggcttggtatggcgtgggtactgccctgaaggaattccttgaggaaaaacccgctgagcatctctccctcttgcgctacttctactataagtggcctttcttccgcatggtgatctctaaggttgagatgacccttgccaaggttgatctagagattgcccgctactatgtccaagaactcagccagccccaaaaccgtgaagccttctgccgcctctacgatcagattgctcaggaatatcgcctgaccacggaattagtcctcacgattactggccatgagcggctactcgatggggatccggcgcttcagcgatcggtgcaactgcgcaatcgcaccattgttcctttgggcttcctgcaagtatctcttttgaaacggctgcgccagcacaatagccaaaccacctctggggcaattttgcgctcccgctatggtcggggtgaattgctacggggggcactcttgaccatcaatggcatagcagcggggatgcgcaatacaggctaagcaacggcgagggtgaatcatggacccgacgacccg

SEQ ID NO: 2 - protein, 1011 aa, Synechococcus vulcanus
MTSVLDVTNRDRLIESESLAARTLQERLRLVEEVLVDVLAAESGQELVDLLRRLGALSSPEGHVLHAPEGELLKVIESLELNEAIRAARAFNLYFQIINIVEQHYEQQYNRERAAQEGLRRRSVMSEPISGVSGEGFPLPHTAANATDVRSGPSERLEHSLYEAIPATQQYGSFAWLFPRLQMLNVPPRHIQKLLDQLDIKLVFTAHPTEIVRQTIRDKQRRVARLLEQLDVLEGASPHLTDWNAQTLRAQLMEEIRLWWRTDELHQFKPEVLDEVEYTLHYFKEVIFAVIPKLYRRLEQSLHETFPALQPPRHRFCRFGSWVGGDRDGNPYVKPEVTWQTACYQRNLVLEEYIKSVERLINLLSLSLHWCDVLPDLLDSLEQDQRQLPSIYEQYAVRYRQEPYRLKLAYVLKRLQNTRDRNRALQTYCIRRNEAEELNNGQFYRHGEEFLAELLLIQRNLKETGLACRELDDLICQVEVFGFNLAALDIRQESTCHAEALNEITAYLGILPCPYTELSEAERTRWLLSELSTRRPLIPGELPFSDRTNEIIETFRMVRQLQQEFGTDLCNTYIISMSHEVSDLLEVLLFAKEAGLFDPATGASTLQAIPLFETVEDLKHAPAVLTQLFSLPFCRSYLGSNSTPFLQEVMLGYSDSNKDSGFLSSNWEIYKAQQQLQKIAESFGFQLRIFHGRGGSVGRGGGPAYAAILAQPAQTIKGRIKITEQGEVLASKYSLPELALFNLETVATAVIQASLLRSSIDEIEPWHEIMEELATRSRQCYRHLIYEQPEFIEFFNEVTPIQEISQLQISSRPTRRGGKKTLESLRAIPWVFSWTQTRFLLPAWYGVGTALKEFLEEKPAEHLSLLRYFYYKWPFFRMVISKVEMTLAKVDLEIARYYVQELSQPQNREAFCRLYDQIAQEYRLTTELVLTITGHERLLDGDPALQRSVQLRNRTIVPLGFLQVSLLKRLRQHNSQTTSGAILRSRYGRGELLRGALLTINGIAAGMRNTG

SEQ ID NO: 3 - DNA, 41 bp, ARTIFICIAL SEQUENCE
  SYNTHETIC DNA
gtcctcgagaatctgaaaaacaatgacatcagtcctcgatg

SEQ ID NO: 4 - DNA, 28 bp, ARTIFICIAL SEQUENCE
  SYNTHETIC DNA
gtcgagctcttagcctgtattgcgcatc

SEQ ID NO: 5 - DNA, 22 bp, ARTIFICIAL SEQUENCE
  SYNTHETIC DNA
gatatctccactgacgtaaggg

SEQ ID NO: 6 - DNA, 25 bp, ARTIFICIAL SEQUENCE
  SYNTHETIC DNA
cccagtcacgacgttgtaaaacgac

(19) United States
(12) Patent Application Publication
(10) Pub. No.: US 2003/0115638 A1
(43) Pub. Date: Jun. 19, 2003

(54) Plants having increased amino acids content and methods for producing the same

(75) Inventors: Katsura Izui, Kyoto-Shi (JP); Tetsuya Miwa, Kawasaki-Shi (JP); Hiroaki Kisaka, Kawasaki-Shi (JP); Ai Akiyama, Kawasaki-Shi (JP)

Correspondence Address: OBLON, SPIVAK, MCCLELLAND, MAIER & NEUSTADT, P.C., 1940 DUKE STREET, ALEXANDRIA, VA, 22314, US

(73) Assignee: AJINOMOTO CO., INC., Tokyo (JP)

(21) Appl. No.: 10/217,411

(22) Filed: Aug. 14, 2002

(30) Foreign Application Priority Data
Aug. 17, 2001 (JP) 2001-247580

(51) Int. Cl. A01H 5/00; C07H 21/04; C12N 9/10; C12N 1/20; C12P 21/02; C12N 5/04

(52) U.S. Cl. 800/287; 435/69.1; 435/193; 435/320.1; 435/419; 435/252.3; 536/23.2

(57) ABSTRACT

The present invention provides a transformed plant having free-amino acid content increased by introducing phosphoenolpyruvate carboxylase (PEPC) genes, and also a progeny thereof and a seed thereof. Namely, the transformed plant has a nucleic acid construct containing PEPC gene introduced therein, wherein phosphoenolpyruvate carboxylase encoded by the PEPC gene does not require phosphorylation for the activation thereof and/or is independent of acetyl CoA. In particular, the present invention provides a transformed plant comprising a nucleic acid construct containing a nucleic acid molecule encoding Synechococcus vulcanus PEPC or a nucleic acid construct containing a nucleic acid molecule encoding a protein having PEPC activity which has at least 80% of nucleotide sequence homology to said gene.

### BACKGROUND OF THE INVENTION

The present invention relates to a transformed plant having increased free amino acids content and a method for producing them.

Plants are capable of synthesizing all the compounds indispensable for living, as they are autotrophs. Amino acids are included in such compounds. Plants synthesize all of 20 naturally occurring amino acids using the light energy from water, carbon dioxide and inorganic nitrogen sources available in the environment. Animals including human beings can not synthesize all of the amino acids and amino acids which cannot be synthesized by the animals are nutritionally important as essential amino acids. Animals primarily depend on amino acids produced by plants for such essential amino acids. Therefore, the improvement in the quality and amount of amino acids contained in plants is an important issue for increasing the nutritional value of the plants.

Further, the increase in the capacity of plants for synthesizing amino acids is also significant from the viewpoint of the growth of the plants themselves. As described above, plants synthesize amino acids from inorganic nitrogen in the environment, which can be recognized as a process of assimilation of nitrogen as amino acids, when considered the meaning in plants. Namely, plants finally assimilate nitrogen in the form of ammonia, into glutamic acid, and glutamic acid is partitioned and utilized as a nitrogen source for various components of living bodies such as other amino acids and nucleic acids. Accordingly, the increase in the capacity of plants for synthesizing amino acids is, in other words, an improvement in nitrogen utilization efficiency of plants. Nitrogen is one of the major limiting factors for the growth of plants. If the capacity of assimilation of nitrogen can be increased as a result of the increase in the amino acid-synthesizing capacity, the acceleration of the growth of the plants is expected and, accordingly, an increase in the yield thereof is also expected. In addition, if nitrogen can be efficiently used, the amount of inorganic nitrogen-containing substances used as fertilizers can be minimized. As a result, an effect of reducing the environmental load is expected.

As a method for increasing amino acids content in plants, enhancing the activity of phosphoenolpyruvate carboxylase may be taken into consideration. Phosphoenolpyruvate carboxylase (hereinafter referred to as PEPC) catalyzes the reaction of producing oxaloacetic acid by fixing bicarbonate into phosphoenolpyruvic acid. This enzyme is widely distributed in microorganisms and plants, and is metabolically located at the junction between glycolytic pathway and TCA cycle. It is said that this enzyme has two physiologically very important roles. One of them is to form a bypass from glycolysis system to TCA cycle so as to provide a carbon backbone to TCA cycle for keeping the turnover of TCA cycle. TCA cycle has a role of generating energy under aerobic conditions and providing starting materials for biosynthesis such as amino acids. Therefore, compounds which constitute TCA cycle may run off from to other metabolism systems, and only a part of carbons can return to the entrance of the cycle where the condensation reaction of oxaloacetic acid and acetyl CoA occurs to produce citrate. Accordingly, for maintaining the turnover of the cycle, it is necessary to compensate the run off carbon, for which PEPC is responsible (it is scientifically called “anaplerotic role”). The enhancement of this enzyme may finally result in the enhancement of carbon supply to TCA cycle and accumulation of compounds derived from TCA cycle, that is, amino acids, is expected.

Another role of this enzyme is the initial carbon dioxide fixation in C4 type photosynthesis of plants. In the C4 type photosynthesis, carbon dioxide fixation occurs through two steps in two types of differentiated cells (mesophyll cells and vascular bundle sheath cells). Namely, carbon dioxide is firstly fixed in malic acid or the like which is transported to vascular cells where released carbon dioxide is fixed in saccharides through the regular carbon fixation reaction. In this type of photosynthesis the concentration of carbon dioxide provided to the carbon fixation is characteristically higher than the regular photosynthesis and the efficiency of carbon oxide fixation will be higher. The regulatory point of this process is believed to reside in the step of carbon dioxide fixation by PEPC. The enhancement of this enzyme is also expected to affect the ability of photosynthesis of plants, in other words, the total capacity of producing materials.

Thus, it was expected to improve amino acid productivity of plants or, in other words, the whole productivity by the enhancement of PEPC. Under these circumstances, it has been tried to introduce PEPC genes from various sources into plants to enhance the enzymatic activity of PEPCs. For example, Johanna Gehlen et al. in Germany introduced PEPC genes of Escherichia coli or Corynebacterium glutamicum into potatoes under the control of Cauliflower Mosaic Virus 35S promoter [Johanna Gehlen, et al., Plant Molecular Biology, 32, 831-848 (1996)]. Uchimiya et al. introduced PEPC of corn into tobacco under the same control as above [Hiroyuki Kogami, et al., Transgenic Research, 3, 287-296 (1994)]. Further, a group of Ministry of Agriculture, Forestry and Fisheries introduced corn PEPC genes in the form of promoter-containing genomic fragment (not in the form of cDNA) into rice plants [Maurice S. B. Ku, et al., Nature Biotechnology, 17, 76-80 (1999) and Mitsuru Osaki, et al., Proceedings of Japanese Society of Plant Physiologists 2000, p. 63]. It is reported therein that a large amount of PEPC protein was accumulated in these transformed plants and that the enzymatic activity was detected in the cell-free extracts of them. However, there was no description reporting that significant phenotypes emerged in these plants and any significant accumulation of amino acids has not been reported.

On the other hand, it is known that 15^{th} and 8^{th} serine residues from the N-terminal of corn PEPC and sorghum PEPC are phosphorylated, respectively. These enzymes are established to be activated by phosphorylation due to the reduction of sensitivity to the allosteric inhibitor (malate) [Jean Vidal and Raymond Chollet, Trends in Plant Science, 2, 230-237 (1997)]. The sequence around the phosphorylated serine is well conserved between maze and sorghum. This sequence is ERLSSIDAQ as indicated in one letter amino acid notation, in which the 5^{th} serine residue in the sequence is phosphorylated. The primary structure of PEPC has been elucidated in many species of plants. Most of them have a sequence similar to this sequence near the N-terminal and the sequence ranging from the 5^{th} serine to the 9^{th} glutamine of the sequences is completely conserved. Accordingly, these enzymes are supposed to require phosphorylation to exhibit their activities.

T Nakamura, et al., [J. Biochem., 120, 518-524 (1996)] reported that the addition of acetyl CoA was indispensable for determining the activity of PEPC from microorganisms and that at least 0.05 mM of acetyl CoA was required in the reaction solution. In fact, when the affinity for phosphoenolpyruvic acid as the substrate in the presence of acetyl CoA at a concentration of 0.05 mM was compared with the affinity in the presence of 0.3 mM acetyl CoA, the affinity was reduced to about ½ of that the latter and, in addition, the expression of the enzymatic activity was also reduced. Further, acetyl CoA concentration in plant cells is reported to be 0.01 to 0.02 mM even chloroplast which is the intracellular small organ for synthesizing fatty acids and which contains a high concentration of acetyl CoA [P. G. Roughan, Biochem. J., 327, 267-273 (1997)].

### SUMMARY OF THE INVENTION

The object of the present invention is to produce transformed plants having a free amino acid content increased by introducing PEPC genes therein.

Another object of the present invention is to provide a progeny of the transformed plant and a seed thereof.

The inventors thought that the amino acid content was not increased even by the introduction of PEPC genes and also by the excessive accumulation of the enzymatic protein thereof because the expressed enzyme protein was in inactive state in the plant cells due to the features of the expressed enzyme protein. The present invention has been completed on the basis that the intended effect would be obtained by introducing the genes encoding PEPC which can express its activity in plant cells.

Namely, the present invention provides a transformed plant having a nucleic acid construct containing a phosphoenolpyruvate carboxylase (PEPC) gene introduced therein, wherein said phosphoenolpyruvate carboxylase encoded by the PEPC gene does not require phosphorylation for the activation thereof and/or said phosphoenolpyruvate carboxylase is acetyl CoA-independent for its activity.

In particular, the present invention provides a transformed plant having a nucleic acid construct containing a phosphoenolpyruvate carboxylase (PEPC) gene introduced therein, wherein said phosphoenolpyruvate carboxylase encoded by the PEPD gene does not require phosphorylation for the activation thereof and said phosphoenolpyruvate carboxylase is acetyl CoA-independent for its activity.

The present invention also relates to a transformed plant having a nucleic acid construct containing PEPC gene from a cyanobacterium.

In particular, the present invention relates to a transformed plant having a nucleic acid construct containing nucleic acid molecule encoding Synechococcus vulcanus PEPC, or a nucleic acid construct encoding a protein having PEPC activity exhibiting the homology in the nucleotide sequence at least 80% to the PEPC gene form Synechococcus vulcanus.

The present invention also includes a seed of the transformed plant.

The present invention further relates to a method for producing a transformed plant having free amino acids content higher than that of an untransformed plant of the same type cultivated under the same condition, which comprises the step of introducing a nucleic acid construct capable of expressing a PEPC gene in plant cells, wherein PEPC encoded by said PEPC gene does not require phosphorylation for the activation thereof and/or said phosphoenolpyruvate carboxylase is acetyl CoA-independent for its activity.

In particular, the present invention relates to a method for producing a transformed plant having free amino acids content higher than that of an untransformed plant of the same type cultivated under the same condition, which comprises the step of introducing a nucleic acid construct capable of expressing PEPC gene into plant cells, wherein PEPC encoded by said PEPC gene does not require phosphorylation for the activation thereof and said phosphoenolpyruvate carboxylase is acetyl CoA-independent for its activity.

The present invention also relates to the above-described method for producing a transformed plant having free amino acids content higher than that of an untransformed plant of the same type cultivated under the same condition, wherein the PEPC gene derives from a cyanobacterium.

In particular, the present invention relates to the above-described method for producing a transformed plant having free amino acids content higher than that of an untransformed plant of the same type cultivated under the same condition, wherein the nucleic acid construct containing a PEPC gene is a nucleic acid construct containing a nucleic acid molecule encoding the PEPC from Synechococcus vulcanus or a nucleic acid construct encoding a protein having PEPC activity exhibiting at least 80% nucleotide sequence homology to the above-described genes.

### BRIEF DESCRIPTION OF THE DRAWINGS

FIG. 1 shows amino acids content (nmole/gFW) per gram of fresh weight of a transformed plant to which Synechococcus vulcanus PEPC genetic construct has been introduced.

FIG. 2 shows the ratio of amino acids content (nmole/gFW) per gram of fresh weight of a transformed plant to which Synechococcus vulcanus PEPC genetic construct has been introduced compared to that of a control plant.

### DETAIL DESCRIPTION OF THE INVENTION

The present invention provides a transformed plant having a nucleic acid construct containing phosphoenolpyruvate carboxylase (PEPC) gene introduced therein, wherein the phosphoenolpyruvate carboxylase encoded by the PEPC gene does not require phosphorylation for the activation thereof and is acetyl CoA-independent for its activity. The term “plant” herein means a whole plant body or a part of a whole plant including leaves, stems, roots, tuberous roots, tubers, fruits and flowers.

In the present invention, PEPC or a gene encoding PEPC, wherein the PEPC does not require phosphorylation for its activation and/or the PEPC is acetyl CoA-independent for its activity.

As described above, it has been reported that corn PEPC and sorghum PEPC are not activated unless serine residue at the N-terminal is phosphorylated [Jean Vidal and Raymond Chollet, Trends in Plant Science, 2, 230-237 (1997)]. Accordingly, it is considered that these enzymes must be phosphorylated for expressing their activities. Generally, plant PEPCs should be phosphorylated for expressing their activities.

The activity of PEPC used in the present invention is not controlled by phosphorylation. In particular, phosphorylation is not necessary for exhibiting the activity. It is particularly preferred that PEPCs used in the present invention do not contain N-terminal conserved phosphorylation sequence or similar sequences thereof in the primary structure.

As described above, plant PEPCs which are activated by phosphorylation usually have the well-conserved amino acid sequence ERLSSIDAQ or similar sequences at the N-terminal. Therefore, PEPCs used in the present invention preferably have not ERLSSIDAQ or similar amino acid sequences in the N-terminal region. The term “amino acid sequence similar to ERLSSIDAQ” as used herein indicates a sequence containing one or more replacement(s) among amino acids which are considered to be similar to each other from the viewpoints of the charge, molecular structure, and the like, including X_{A}-X_{B}-X-X-SIDAQ, wherein X_{A} represents an acidic amino acid, X_{B} represents a basic amino acid and X represents an amino acid.

PEPCs used in the present invention are acetyl CoA-independent in expressing their activities. As described above, because PEPCs which do not require phosphorylation for their activation are suitable for the purpose of the present invention, PEPC genes from other sources than plants are preferred. However, although microorganism-derived PEPCs are considered to require no phosphorylation for expressing their activities and it is expected that the activity is expressed in the cells, the expected effects could not be actually observed by introducing PEPC genes derived from E. coli or Corynebacterium. The inventors supposed that the cause for this phenomenon was that the expression of the activity of PEPC of microorganism origin was dependent on acetyl CoA. As described above, PEPCs from microorganism require at least 0.05 mM of acetyl CoA in vitro. Further, it was reported that even when the concentration of acetyl CoA was 0.05 mM, the affinity for phosphoenolpyruvic acid used as the substrate was reduced to about ½ of that obtained when the concentration of acetyl CoA is 0.3 mM and, in addition, the expression of the enzymatic activity was also reduced [T. Nakamura, et al., J. Biochem., 120, 518-524 (1996)]. Such a dependency of PEPC on acetyl CoA is considered to exert an influence on the expression of the activity thereof also in vivo.

On the other hand, acetyl CoA concentration in plant cells is considered to be 0.01 to 0.02 mM even in chloroplast which is the intracellular small organ for synthesizing fatty acids and which contains a high concentration of acetyl CoA [P. G. Roughan, Biochem. J., 327, 267-273 (1997)]. Therefore, it is supposed that even when PEPC of microorganism origin is present as an enzyme protein, the activity would be scarcely expressed. PEPC used in the present invention is thus preferably acetyl CoA-independent. The expression “PEPC is acetyl CoA-independent” means that the PEPC exhibits its activity even in the absence of acetyl CoA or in the presence of less than 0.05 mM of acetyl CoA in vitro, preferably the PEPC exhibits its activity in the presence of 0.05 mM of acetyl CoA in vitro, at least 70%, more preferably at least 80% of the activity obtained at about the optimum concentration thereof in vitro.

PEPCs used in the present invention are desirably heat resistant. Because a protein from a different species tends to become unstable in a heterologous host, it is supposed that the expression of an essentially stable protein is advantageous for the expression of the enzymatic activity. The term “heat resistant” herein indicates the property that the activity at a temperature of 40° C. or higher is at least half of the activity observed at optimum temperature.

PEPCs usable in the present invention are, for example, PEPCs from cyanobacteria. Cyanobacteria are defined as organisms classified as plants, which do not have membrane-enveloped nuclei or chloroplasts. Cyanobacteria are substantially the synonym for bacteria having photosynthetic ability. PEPC genes are isolated from various species of organisms classified as cyanobacteria such as single-cell cyanobacteria, e.g. Synechocystis nidulans, filamentous cyanobacteria, e.g. Anabaena valiabilis and heat-resistant cyanobacteria, e.g. Synechococcus valcanus, and the structures of them were elucidated. They do not have ERLSSIDAQ or a similar sequence near the N-terminal thereof [H. Toh et al., Plant, Cell and Environment, 17, 31-43 (1994), L-M Chen and K. Izui, Proceedings of Japanese Society of Plant Physiology 2000, p. 158]. Further, the activity of Synechocystis nidulans [T. Kodaki, et al., J. Biochem. 97, 533-539 (1985)], Synechococcus vulcanus [L-M Chen and K. Izui, Purports of Proceedings of Japanese Society of Plant Physiology 2000, p. 158] and Coccochloris peniocystis [G. W. Owttrim and B. Colman, J. Bacteriol., 168, 207-212 (1986)] was detected even in the absence of acetyl CoA, which indicates that they are acetyl CoA-independent. Namely, cyanobacteria PEPCs generally have the properties of PEPC usable in the present invention. Particularly, the sources of PEPCs and PEPC genes used in the present invention are preferably cyanobacteria belonging to the genus Synechococcus, more preferably Synechococcus vulcanus. In a preferred embodiment of the present invention, PEPC genes and PEPCs from Synechococcus vulcanus are used. The sequence of PEPC gene from Synechococcus vulcanus is shown in SEQ ID NO: 1, and PEPC amino acid sequence encoded by this gene is shown in SEQ ID NO: 2.

Synechococcus vulcanus is taxonomically comparable to Synechococcus elongatus and Synechococcus lividus. These microorganisms can be obtained from American Type Culture Collection (ATCC). Accession numbers of them are ATCC 27184, ATCC 33912 and ATCC 27179, respectively.

The PEPC activity, the lack of requirement of phosphorylation for activation and the independency of acetyl CoA are the properties which depend on the gene sequence and, in other words, on the amino acid sequence thereof. Accordingly, proteins having the amino acid sequence homologous to that of PEPC from Synechococcus vulcanus are considered to be usable in the present invention. Nucleic acid molecules encoding such proteins can be obtained as molecules having the homology of at least 80%, preferably at least 90% and more preferably at least 95%, at the nucleotide sequence level, as calculated using a program which is capable of calculating the sequence homology such as Blast with the standard parameters. Further, nucleic acid molecules encoding the amino acid sequences homologous to Synechococcus vulcanus PEPC can be obtained as nucleic acid molecules capable of hybridizing under a stringent condition with the nucleic acid molecule encoding Synechococcus vulcanus PEPC. The stringent condition may be determined, for example, from the knowledge of that the melting temperature (Tm) of double strand DNA can be determined on the basis of the finding that Tm value is reduced under the ordinary hybridization condition by 1 to 1.5° C. from the value calculated according to the following well-known formula as the homology decreases by 1%:

Tm=81.5° C.+16.6(log₁₀[Na⁺])+0.41(GC content)−0.63 (formamide % concentration)−(600/l)

wherein [Na⁺] represents sodium ion concentration, and l represents the length of the sequence.

Preferably, PEPC having an amino acid sequence homologous to that of PEPC from Synechococcus vulcanus has the PEPC activity, it does not require phosphorylation for the activation thereof and the activity thereof is acetyl CoA-independent. The lack of requirement of phosphorylation for activation may be easily confirmed by, for example, the fact that the PEPC does not have an amino acid sequence similar to ERLSSIDAQ at the N-terminal. The relationship between phosphorylation and the activity can be directly determined. The fact that the activation is acetyl CoA-independent can be easily confirmed by determining PEPC activity in the absence of acetyl CoA or in the presence of various concentrations of acetyl CoA.

PEPC activity can be determined by various methods. Ordinary methods for determining PEPC activity comprise coupling PEPC with malate dehydrogenase and determining thus obtained oxaloacetic acid in terms of a reduction in the absorbance of reduced nicotinamide nucleotide. Namely, the purified enzyme or cell-free extract containing the enzyme is added to a reaction solution containing 2 mM PEP, 10 mM KHCO₃, 10 mM MgSO₄, 0.1 mM NADH, 0.1 M Tris-HCl (pH 8.6) and 1.0u porcine heart malate dehydrogenase (available from, for example, Sigma-Aldrich Co.), and the mixture is heated to various degrees of temperature. In this step, oxaloacetic acid formed by the reaction of PEPC is quantitatively reduced by malate dehydrogenase and, accordingly, NADH which is a coenzyme of malate dehydrogenase is oxidized and thereby decreases in the quantity. Because NADH has a specific absorption at a wavelength of 340 nm, the decrease in quantity of NADH can be determined in terms of the reduction in the absorbance at a wavelength of 340 nm with a spectrophotometer. PEPC activity can be thus determined [T. Nakamura, et al., J. Biochem, 120, 518-524 (1996)]. By such a process, acetyl CoA independency of the enzyme can be confirmed by comparing the activity observed in the presence of 0 to about 0.05 mM of acetyl CoA with the activity observed in the presence of an acetyl CoA in a concentration considered to be required for obtaining a sufficient activity such as the maximum activity, for example, about 0.3 mM of acetyl CoA.

In the present invention, a plant having an increased total free amino acids content, in particular, free asparagine, glutamine and arginine content, can be obtained by transforming a plant with a nucleic acid construct containing PEPC gene which does not require phosphorylation for the activation and/or which is acetyl CoA-independent and expressing the gene. Thus the transformed plant can be obtained in a preferred embodiment of the present invention which has total free amino acids content increased to at least 4 times as high; an asparagine content increased to at least 3 times as high and desirably at least 4 times as high; the glutamine content increased to at least 8 times as high, desirably at least 10 times as high and more desirably at least 16 times as high; and arginine content increased to at least 4 times as high, desirably at least 5 times as high and more desirably at least 6 times as high.

The nucleic acid constructs used in the present invention can be obtained by methods well known in the art. As for the molecular biological techniques for isolating nucleic acid constructs and determining the sequences thereof, literatures such as Sambrook, et al., Molecular cloning-Laboratory manual, 2^{nd} edition (Cold Spring Harbor Laboratory Press) can be referred to. The gene amplification by PCR method and the like may be required in some cases for the production of the nucleic acid constructs usable in the present invention. For such techniques, Current Protocols in Molecular Biology edited by F. M. Ausubel, et al. and published by John Wiley & Sons, Inc. (1994) can be referred to.

The nucleic acid constructs usable in the present invention may contain a suitable promoter, which functions generally in plants or in plant cells as part of plants, such as nopaline synthase genes, 35S promoter for cauliflower mosaic virus (CaMV35S), a suitable terminator such as the terminator of nopaline synthase gene, other sequences necessary or advantageous for the expression, and marker genes for selecting the transformant such as drug resistant genes, e.g. genes resistant to kanamycin, G418 or hygromycin.

The promoters usable for the constructs may be either constitutive promoters or organ-specific or growing stage-specific promoters. The promoters can be selected depending on the host to be used, the required expression level, the organ in which the expression is particularly intended or the growing stage. In a preferred embodiment of the present invention, a strong promoter which expresses non-specifically in the organs or growing stage is used. Such promoters include, for example, CaMV35S promoter. The organ specific promoters include phaseolin gene promoter, patatin gene promoter, etc. In the most preferred embodiment of the present invention, a construct capable of driving the PEPC genes with a powerful constitutive promoter such as CaMV35S promoter is used.

The method for introducing the genes is not particularly limited in the present invention. Any method known by those skilled in the art for introducing genes into plant cells or into the plant body can be selected depending on the host. For example, in an embodiment of the present invention, the gene introduction method using Agrobacterium is employed. In such a transformation system, binary vectors are preferably used. When Agrobacterium is used, the nucleic acid construct used for the transformation further contains at least the right border sequence in T-DNA range adjacent to the DNA sequence to be introduced into the plant cells. In a preferred embodiment, the introduced sequence is inserted between the left and right T-DNA border sequences. The suitable design and construction of transformation vectors based on T-DNA are well known in the art. Further, the conditions required for infecting plants with Agrobacterium harboring such a nucleic acid construct are also well known in the art. As for such techniques and conditions, “Model Shokubutsu no Jikken Protocol, Ine, Shiroinunazuna Hen (Experiment Protocol for Model Plants; Rice Plants and Arabidopsis thaliana) (1996) can be referred to.

In the present invention, other gene introduction methods can also be used. Examples of the gene introduction methods which can be employed herein include the method for introducing DNA into protoplasts with polyethylene glycol or calcium, the method for transforming protoplasts by electroporation, the method using a particle gun, etc.

Although the species of plants to be subjected to the genetic manipulation as described above are not particularly limited, those which are easily transformed and the regeneration system of which has been established are preferred when the plant bodies per se are used for the manipulation. In addition to the plants having the above-described characteristic properties, plants species for which a large-scale cultivation techniques have been established are preferred in the present invention from the viewpoint of the utilization of produced amino acids. Plants suitable for the method of the present invention include, for example, Brassicaceae as well as tomatoes, potatoes, corn, wheat, rice plant, sugarcane, soybean and sorghum. The organs and cells which are subjected to the above-described genetic manipulation are not particularly limited, and they can be selected depending on the host, gene introducing method, etc. Examples of them include, but are not limited to, explants, pollens, cultured cells, embryos and plant bodies.

Then the manipulated plant cells and the like undergo the selection for transformation. The selection may be based on the expression of a marker gene located on the nucleic acid construct used for the transformation. For example, when the marker gene is a drug resistant gene, the selection can be conducted by culturing or growing thus manipulated plant cells and the like on a culture medium containing a suitable concentration of an antibiotic or a herbicide. When the marker gene is β-glucuronidase gene or luciferase gene, the transformants can be selected by screening those having the activity. When thus identified transformed plants are not whole plant bodies, in other words, when they are protoplasts, calli or explants, the regeneration of plants may be performed. A method for regeneration known by those skilled in the art for each host plant can be employed. The plants thus obtained can be cultivated by an ordinary method or, in other words, under the same conditions as those for the untransformed plants. For the identification of the transformed plants containing the nucleic acid constructs of the present invention, various molecular biological methods can be employed in addition to the above-described marker gene selection method. For example, Southern hybridization or PCR can be employed for detecting the inserted recombinant DNA segments and also the structure thereof. Northern hybridization or RT-PCR can be employed for detecting and determining RNA transcripts from the introduced nucleic acid construct.

The expression of PEPC genes of the obtained transformants can be evaluated on the basis of the amount of the PEPC protein, amount of mRNA or the activity in the cell-free extracts from the transformed plants. For example, the amount of PEPC protein can be determined by Western blotting method or the like, and the amount of mRNA can be determined by Northern blotting method or quantitative RT-PCR method. PEPC activity can be determined by, for example, the method of Nakamura et al. [T. Nakamura, et al., J. Biochem., 120, 518-524 (1996)]. It is possible to examine the sensitivity of PEPC activity to various effectors, particularly acetyl CoA dependency. These methods are well known in the art, and kits for performing them may be commercially available.

After the expression of the PEPC gene in the transformed plant is thus confirmed, free amino acids content of the plant is further determined. The free amino acids content can be examined by, for example, crushing the transformed plant or a part thereof, obtaining an extract and examining the extract with an amino acid analyzer. The transgenic plants can be cultivated under the similar conditions as untransformed plants. For example, in a laboratory test, a culture medium containing ½ MS salt and ½ B5 salt, 10 g/l sucrose and 0.8% agar (pH is adjusted to 5.5 with KOH) or PNS medium containing 5 mM KNO₃, 2.5 mM KH₂PO₄, 2.0 mM MgSO₄, 2.0 mM Ca(NO₃)₂, 0.05 mM Fe-EDTA, 0.07 mM H₃BO₃, 0.014 mM MnCl₂, 0.0005 mM CuSO₄, 0.001 mM ZnSO₄, 0.0002 mM Na₂MoO₄, 0.01 mM NaCl, 0.00001 mM CoCl₂ (pH: adjusted to 5.5 with KOH), 10 g/l sucrose and 0.8% agar can be used.

MS medium contains 1650 mg/l NH₄NO₃, 1900 mg/l KNO₃, 440 mg/l CaCl₂·2H₂O, 370 mg/l MgSO₄·7H₂O, 170 mg/l KH₂PO₄, 6.2 mg/l H₃BO₃, 22.3 mg/l MnSO₄·4H₂O, 8.6 mg/l ZnSO₄·7H₂O, 0.83 mg/l KI, 0.25 mg/l Na₂MoO₄·2H₂O, 0.025 mg/l CuSO₄·5H₂O, 0.025 mg/l CoCl₂·6H₂O, 37.3 mg/l Na₂·EDTA·2H₂O, 27.8 mg/l FeSO₄·7H₂O as salts. B5 medium contains 2500 mg/l KNO₃, 250 mg/l MgSO₄·7H₂O, 150 mg/l NaH₂PO₄·H₂O, 150 mg/l CaCl₂·2H₂O, 134 mg/l (NH₄)₂SO₄, 37.3 mg/l Na₂·EDTA·2H₂O, 27.8 mg/l FeSO₄·7H₂O, 10 mg/l MnSO₄·H₂O, 3 mg/l H₃BO₃, 2 mg/l ZnSO₄·7H₂O, 0.75 mg/l KI, 0.25 mg/l Na₂MoO₄·2H₂O, 0.025 mg/l CuSO₄·5H₂O, 0.025 mg/l CoCl₂·6H₂O as salts.

After the transformed plants having an increased free amino acids content are thus identified, it is possible to examine whether the properties thereof can be genetically stably maintained or not. For this purpose, the plants are grown or cultivated under ordinary conditions, the seeds are taken from them and the character and segregation of the descendants thereof are analyzed. The presence or absence of the induced nucleic acid constructs, the position and the expression thereof in the progenies can be analyzed in the same manner as that for the primary transformants.

The transformants having an increased free amino acids content are either hemizygous or homozygous as for the sequence derived from the nucleic acid constructs integrated into their genomes. If necessary, either hemizygotes or homozygotes can be obtained by crossing them. The sequences derived from the nucleic acid constructs integrated into the genomes will segregate according to Mendelian in the progenies. Therefore, for obtaining the descendant plants and seeds thereof, it is preferred to use homozygous plants from the viewpoint of the stability of the properties. The transformed plants thus obtained can be grown under the same cultivation conditions as those of the natural plants to provide the crops having an increased amino acids content.

### EXAMPLES

### Example 1

### Integration of Synechococcus vulcanus PEPC Gene into Plant Transformation Vector

The integration of PEPC gene from E. coli expression plasmid SVPPC/pTV, which was designed for Synechococcus vulcanus PEPC expression, into plant transformation vector pBI121Kex was carried out as follows:

pBI121KEx is the vector obtained by replacing the original E. coli β-glucuronidase gene region of pBI121 (product of Clonetech) with a multicloning site containing EcoRI, BamHI, XhoI, NotI and SacI site. Namely, in the structure of pBI121KEx, the region between CaMV35S promoter and the terminator of nopaline synthase is replaced with the above-described restriction site-containing synthetic DNA. Because Synechococcus vulcanus PEPC gene has no cleavage site for XhoI and SacI in its coding region, the integration into pBI121KEx was carried out using these restriction enzymes.

At first, PCR was carried out using SVPPC/pTV or chromosomal DNA as the template with SVPPC-A primer [(5′-GTCCTCGAGaatctgaaaa acaATGACATCAGTCCTCGATG-3′ (SEQ ID NO: 3)) and SVPPC-B primer [5′-GTCGAGCTCTTAGCCTGTATTGCGCATC-3′ (SEQ ID NO: 4)] to obtain PEPC gene fragment with additional sites at both ends for introducing it into pBI121KEx. SVPPC-A was composed of, from 5′ side, three (3) extra bases (for increasing the cleavage efficiency by the restriction enzyme), XhoI recognition sequence, translation acceleration sequence for plants (Kozak box) and a 19-base sequence starting with initiation codon of PEPC gene. SVPPC-B was composed of, from 5′ side three (3) extra bases, SacI recognition sequence and a complementary strand of a 19-base sequence starting with the termination codon of PEPC gene. PCR reaction was carried out with error-free Native Pfu DNA polymerase (the product of Stratagene Co.) under the reaction condition recommended by the supplier. The thus obtained fragment of about 3 kb was purified using PCR purification kit (QIAGEN Co.) and immediately digested with SacI and XhoI. After the digestion followed by the gel filtration through Microspin Column S-300 (Amersham-Pharmacia Co.), the obtained product was subjected to the ligation reaction with pBI121KEx which had been also digested with SacI and XhoI. E. coli was transformed with the ligation mixture by an ordinary method to obtain kanamycin-resistant colonies. These colonies were screened by colony PCR with primer 35S-5D [5′-gatatctccactgacgtaaggg-3′ (SEQ ID NO: 5)] derived from the promoter sequence of CaMV35S and primer NOST-3 [5′-cccagtcacgacgttgtaaacgac-3′ (SEQ ID NO: 6)] derived from the terminator sequence of nopaline synthase to select clones having about 3 kb fragment. Thus, the clones containing the plant transforming plasmid pKExSVPPC were obtained. The fragment amplified by colony PCR was subjected to the direct sequencing using 35S-5D and NOST-3 primers to confirm the sequence.

<Sequence Listing Free Text>

Sequence Nos. 3 and 4: PCR primer for Synechococcus vulcanus PEPC

Sequence Nos. 5 and 6: PCR primer

### Example 2

### Introduction of Synechococcus vulcanus PEPC Gene into Arabidopsis thaliana

The plasmid pKExSVPPC was introduced into Agrobacterium C58C1Rif by triparental mating using E. coli harboring pKExSVPPC and helper E. coli HB101/pRK203. Arabidopsis thaliana Columbia was infected by vacuum infiltration method with thus obtained Agrobacterium C58C1Rif containing pKExSVPPC. The vacuum infiltration method was conducted by the method described in “Model Shokubutsu no Jikken Protocol; Ine, Shiroinunazuna Hen (Experiment Protocol for Model Plants; Edition of Rice Plants and Arabidopsis thaliana)” as the special number of “Saibou Kogaku (Cell Technology)” (published by Shujunsha). The seeds (T1) obtained from the infected plants were planted on GM agar medium [½xMS, 1xB5 vitamin, 10 g/l sucrose, 0.5 g/l MES-KOH (pH 7.5) and 0.8% agar] containing 100 mg/ml of kanamycin after sterilized with a sodium hypochlorite solution having an effective chlorine concentration of 1%, and the transformants were screened.

In many cases, the transformant has one copy of the introduced gene, but it is not rare that the transformant may contain multi-copy of the gene integrated in plural gene loci. Multi-copy transformants are not preferred because their progenies have a problem in the stability of the introduced gene. Thus, a transformant having the gene introduced in one locus must be selected by examining the segregation ratio for kanamycin resistance in T2. In T1 generation, the transformants are hemizygous and, therefore, when the gene is introduced into one locus, the segregation ratio of kanamycin resistant to the kanamycin sensitive plants is 3:1 according to Mendelian in T2 generation. When multi-copy genes are present, the frequency of the resistance is increased. Accordingly, the obtained T2 seeds were again placed on kanamycin-containing media, and the lines having the segregation ratio of 3:1 were selected to obtain transformants in which the gene was considered to have been inserted in one locus of each line.

T3 seeds were further obtained from a part of T2 plants which are resistant to kanamycin. The segregation ratio of the drug resistant was determined and lines from which the sensitive individuals were no more segregated were selected to obtain the lines wherein the introduced gene had become homozygous.

### Example 3

### Amino Acid Analysis of Arabidopsis thaliana Containing Synechococcus vulcanus PEPC Gene

The amino acid analysis of thus transformed Arabidopsis thaliana was conducted.

Seeds of Arabidopsis thaliana were placed on the medium containing ½ MS salt+½ B5 salt+10 g/l sucrose and 0.8% Agar (pH is adjusted to 5.5 with KOH). The seeds were cultivated at 22° C. under the long-day condition consisting of 16 hours of light period and 8 hours of dark period for about 2 weeks. Thus, the seedlings were obtained having about 5 or 6 foliage leaves.

The obtained seedlings were crushed with a mortar and a pestle and were extracted with 80% ethanol at 70° C. then with ether to remove lipid-soluble components. The aqueous layer was freeze-dried and then dissolved in 10 mM HCl to obtain a sample for amino acid analysis. The sample was analyzed for quantifying free amino acids content by an amino acid analyzer LC 8800 (Hitachi, Ltd.).

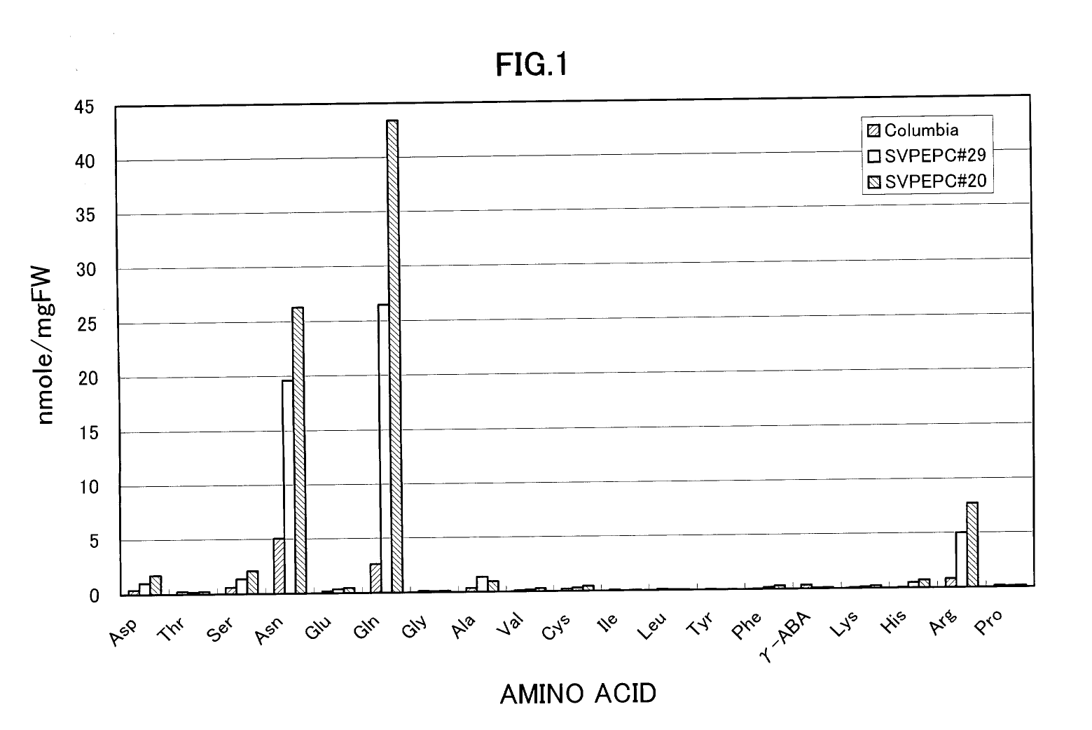

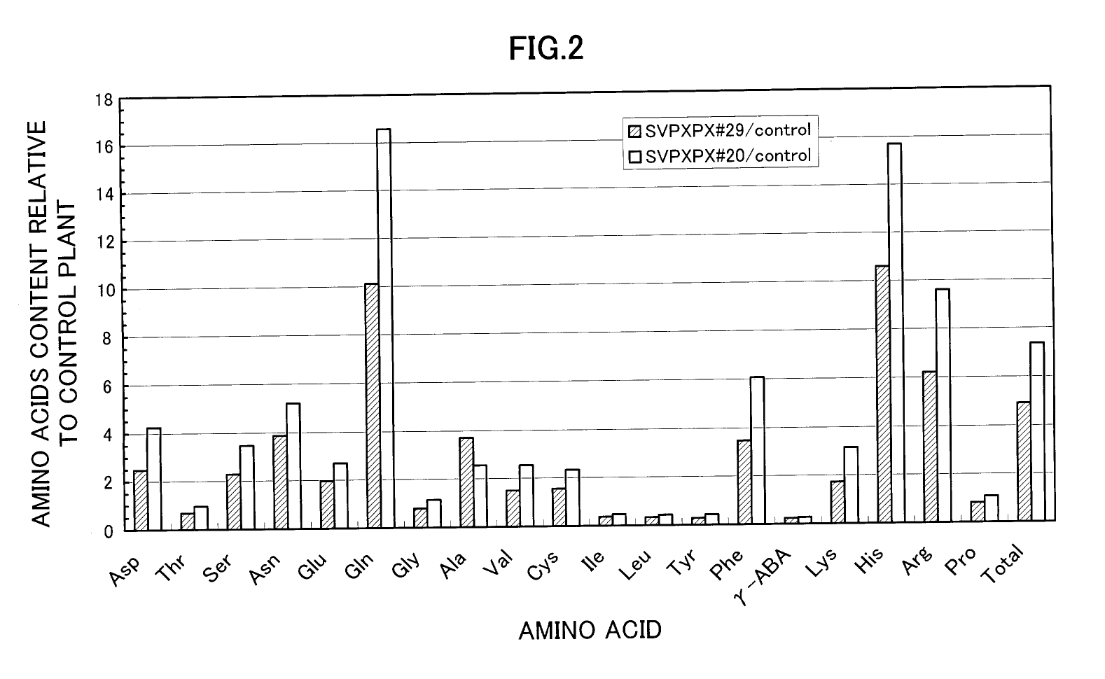

The typical results thus obtained are shown in Table 1 and FIG. 1. The relative amino acids content to the amino acids content of a control plant (untransformed plant) is shown in FIG. 2.

TABLE 1
Amino acids content of transformed plant (nmole/gFW)

| | | SVPEPC-introduced | SVPEPC-introduced |
|---|---|---|---|
| | Control Plant | line #29 | line #20 |
| Asp | 0.41341 | 1.02809 | 1.75879 |
| Thr | 0.23457 | 0.16203 | 0.22321 |
| Ser | 0.60284 | 1.37988 | 2.08933 |
| Asn | 5.05480 | 19.59530 | 26.30475 |
| Glu | 0.18375 | 0.36169 | 0.49758 |
| Gln | 2.61763 | 26.45975 | 43.40750 |
| Gly | 0.11268 | 0.08839 | 0.12794 |
| Ala | 0.37335 | 1.38277 | 0.95714 |
| Val | 0.11027 | 0.16536 | 0.28052 |
| Cys | 0.20003 | 0.31014 | 0.46866 |
| Ile | 0.08162 | 0.02979 | 0.03826 |
| Leu | 0.09700 | 0.03164 | 0.04070 |
| Tyr | 0.04635 | 0.01290 | 0.01961 |
| Phe | 0.04985 | 0.17345 | 0.30295 |
| γ-ABA | 0.34771 | 0.08267 | 0.09413 |
| Lys | 0.07046 | 0.12145 | 0.22166 |
| His | 0.04544 | 0.48136 | 0.71258 |
| Arg | 0.80262 | 4.98573 | 7.70865 |
| Pro | 0.13559 | 0.10958 | 0.14514 |
| Total | 11.57996 | 56.96197 | 85.39909 |

Thus, it was found that the total amino acids content of the plant was remarkably increased by the introduction of Synechococcus vulcanus PEPC genes. While many amino acids were increased in amount, the increase in asparagine, glutamine and arginine was remarkable.

According to the present invention, transformed plants having increased total free amino acids content, especially having increased free glutamine content, free asparagine content or free arginine content, are obtained. In particular, according to the present invention, transformed plants can be obtained in which free glutamine content is increased to about 10 times or more.

What is claimed is:

1. A transformed plant containing phosphoenolpyruvate carboxylase gene introduced therein, wherein said phosphoenolpyruvate carboxylase encoded by the gene does not require phosphorylation for the activation thereof and/or said phosphoenolpyruvate carboxylase is independent of acetyl CoA for its activity.

2. A transformed plant containing phosphoenolpyruvate carboxylase gene introduced therein, wherein said phosphoenolpyruvate carboxylase encoded by the gene does not require phosphorylation for the activation thereof and said phosphoenolpyruvate carboxylase is independent of acetyl CoA for its activity.

3. A transformed plant having phosphoenolpyruvate carboxylase gene introduced therein, wherein said phosphoenolpyruvate carboxylase gene is derived from cyanobacteria.

4. The transformed plant according to claim 1, wherein the phosphoenolpyruvate carboxylase is heat resistant.

5. The transformed plant according to claim 3, wherein the phosphoenolpyruvate carboxylase gene is derived from cyanobacterium belonging to the genus Synechococcus.

6. The transformed plant according to claim 5, wherein the phosphoenolpyruvate carboxylase gene is derived from Synechococcus vulcanus.

7. A transformed plant containing a nucleic acid construct containing a nucleic acid molecule having at least 80% nucleotide sequence homology to Synechococcus vulcanus PEPC gene.

8. A Seed of the plant according to claim 1 comprising the phosphoenolpyruvate carboxylase gene.

9. A Food containing the plant according to claim 1.

10. A method for producing a transformed plant having free-amino acids content higher than that of an untransformed plant of the same type cultivated under the same condition, which comprises the step of introducing a nucleic acid construct containing phosphoenolpyruvate carboxylase gene into a plant, wherein phosphoenolpyruvate carboxylase encoded by the gene does not require phosphorylation for the activation thereof and/or said phosphoenolpyruvate carboxylase is independent of acetyl CoA for its activity.

11. A method for producing a transformed plant having free-amino acids content higher than that of an untransformed plant of the same type cultivated under the same condition, which comprises the step of introducing a nucleic acid construct containing phosphoenolpyruvate carboxylase gene into a plant, wherein phosphoenolpyruvate carboxylase encoded by the gene does not require phosphorylation for the activation thereof and said phosphoenolpyruvate carboxylase is independent of acetyl CoA for its activity.

12. A method for producing a transformed plant having a free amino acids content higher than that of a natural plant of the same kind cultivated under the same condition, which comprises the step of introducing a nucleic acid construct containing phosphoenolpyruvate carboxylase gene into a plant, wherein said phosphoenolpyruvate carboxylase gene is derived from cyanobacterium.

13. The method according to claim 10, wherein the phosphoenolpyruvate carboxylase is heat resistant.

14. The method according to claim 12, wherein the phosphoenolpyruvate carboxylase gene is derived from cyanobacteria belonging to the genus Synechococcus.

15. The method according to claim 14, wherein the phosphoenolpyruvate carboxylase gene is derived from Synechococcus vulcanus.

16. A method for producing a transformed plant having a free-amino acids content higher than that of an untransformed plant of the same type cultivated under the same condition, which comprises the step of introducing a nucleic acid construct containing a nucleic acid molecule having at least 80% nucleotide sequence homology to Synechococcus vulcanus PEPC gene.
